(19) United States
(12) Patent Application Publication
(10) Pub. No.: US 2025/0144370 A1
(43) Pub. Date: May 8, 2025

(54) CANNULA STABILIZATION DEVICE

(71) Applicant: DelStar Technologies, Inc., Alpharetta, GA (US)

(72) Inventors: Nick Shaffer, Alpharetta, GA (US); Tina Anjonrin-Ohu, Alpharetta, GA (US)

(21) Appl. No.: 18/935,183

(22) Filed: Nov. 1, 2024

Related U.S. Application Data
(60) Provisional application No. 63/595,555, filed on Nov. 2, 2023.

(51) Int. Cl. A61M 25/02

(52) U.S. Cl. CPC A61M 25/02; A61M 2025/0253; A61M 2025/0266

(57) ABSTRACT

A securement or stabilization device for holding a cannula or catheter to a patient. The stabilization device may be configured as a universal holding device, and be able to accommodate tubes of varying sizes and shapes, to eliminate the need for one-to-one pairing of the cannula or catheter to its corresponding shaped and sized holder. The stabilization device allows the cannula or catheter to be secured at a desired angle and for an extended period to a patient's skin.

### CROSS-REFERENCE TO RELATED APPLICATIONS

This application claims the benefit of U.S. Provisional Application No. 63/595,555, filed Nov. 2, 2023, the complete disclosure of which is incorporated herein by reference for all purposes.

### TECHNICAL FIELD

The present application relates to holding devices for securing a medical tube to the skin of a patient. More specifically, a securement or stabilization device configured to hold a cannula, catheter or other medical tube in a desired angle to the skin of a patient, such as for use in administering an intravenous drip, is described.

### BACKGROUND

Cannulas and catheters are flexible tubes that can be inserted into a vein or body cavity of a patient to either deliver fluids, medications and other therapies, or remove substances. A peripheral intravenous (IV) catheter or cannula is one such example of these types of intravenous devices that are used to administer or withdraw liquids through blood vessels in a human or animal body. The devices, which typically include a needle for insertion into a blood vessel and a cannula hub (from which the needle extends), are often secured to the patient's skin during the period the liquid is administered or withdrawn. Most often, the cannula is secured to the skin with medical tape. While this is effective for a short period and an inexpensive solution, it also has its disadvantages. Poor securing of a cannula with medical tape can result in it pistoning in and out of the skin, bruising, or the needle becoming dislodged, all of which results in either unneeded pain to the patient or improper delivery or withdrawal of fluids. When a cannula is intended to remain in position for a longer period, it must also be secured to the patient more effectively to reduce the risk of movement of the needle, damage to the patient (e.g., bruising) and dislodgement.

Devices are known and available that can more securely hold a cannula in place. Some are made of relatively stiff materials and others of more flexible materials. A disadvantage of these commercially available devices is that they are each often tailored to a cannula of a certain size and shape. This means that a medical practitioner will need to store a range of securement devices to accommodate each of the different types of cannulas they store, and to select the appropriate one when needed. If the wrong size of securement device is chosen, the cannula will not be fixed very securely in place, leading to the problems mentioned above.

There is a need for a securement or stabilization device that would allow use with cannulas of different sizes and shapes, while still being effective in holding them securely and being cost-effective to make and use.

### SUMMARY

A securement or stabilization device for holding a cannula or catheter to a patient is described herein. The stabilization device may be configured as a universal holding device, and be able to accommodate tubes of varying sizes and shapes, to eliminate the need for one-to-one pairing of the cannula or catheter to its corresponding shaped and sized holder, which is time consuming and poses the above-mentioned risks if an improper pairing is made.

In a first aspect, a stabilization device for a cannula is provided. The stabilization device can comprise first and second portions, a first side and an opposing second side, and a first edge and opposing second edge. The stabilization device can also comprise a compressible pad having a tapered notch, the notch having a wide end and a narrow end, wherein the wide end is disposed at an edge of the compressible pad and at the first edge of the device between the first and second portions. An adhesive may be disposed on the second side at least on the first and second portions of the stabilization device. A securement strip may also be attached to the first side of the first portion of the stabilization device, wherein the securement strip is configured to extend over the notch in the compressible pad to the second portion, and is attachable to the first side of the second portion of the stabilization device.

In an embodiment, the stabilization device may comprise a backing strip, and the first and second portions, first side and opposing second side, and first and opposing second edges of the stabilization device are part of the backing strip. The compressible pad may be disposed on the first side of the backing strip, and the adhesive may be disposed on the second side of the backing strip for adhesion to a skin of a patient.

In an embodiment, the first and second portions, first side and the opposing second side, and first and second edges of the stabilization device are part of the compressible pad, and the adhesive is disposed on the second side of the first and second portions of the compressible pad for adhesion to skin of a patient.

The notch may have an angle of between 5 to 140 degrees, or between 30 to 60 degrees, or between 30 to 50 degrees, and may have a rounded narrow end.

The compressible pad may have a depth and a width, and the ratio of the depth of the compressible pad to the width of the wide end of the notch can be 1:1 to 1:5, or 1:2 to 1:4, or 1:3.

The compressible pad can comprise a material selected from foam, fabric and a compressible non-porous material. The materials of the compressible pad can comprise a polymer selected from polyalkylene (e.g. polyethylene, polypropylene), halogenated polyalkylene (e.g. chlorosulphonated polyethylene), silicone, ethylene vinyl alcohol polymer, polychloroprene (neoprene), styrene butadiene, polystyrene, natural rubber, butyl rubber, nitrile rubber, hydrogenated nitrile, ethylene propylene diene, fluoroelastomer rubber, polyurethane (e.g. a microcellular polyurethane), polyurea, polyimide, polyvinylchloride, hydrocolloid and hydrogel. The compressible pad can comprise a closed cell foam.

The backing strip can comprise a continuous web of material, a fabric, a foam, a paper, and polymeric material.

The adhesive can be selected from a silicone, an acrylic, a hydrocolloid, a hydrogel, a rubber adhesive and a polyurethane.

The stabilization device can also include a release liner on the adhesive.

In an embodiment, the compressible pad can comprise two layers, and the tapered notch can be a stepped notch.

In a second aspect, a method of securing a cannula to a patient is provided. The method may include the steps of inserting a needle of a cannula into a blood vessel of a patient, adhering the stabilization device on a skin of the patient, placing the cannula into the tapered notch of the stabilization device such that the needle extends from away from the wide end of the notch, and extending the securement strip over the cannula and the notch, such that it is adhered to the first side of both the first and second portions of the stabilization device.

In an embodiment, the cannula can comprise a cannula hub from which the needle of the cannula extends, and the cannula hub can be placed in the tapered notch.

In an embodiment, the cannula can comprise tubing, and the tubing can be placed in the tapered notch.

The cannula stabilization device as described herein can be used with cannulas of different sizes and shapes, while still allowing them to be secured securely and for an extended period to a patient's skin. Cannulas, as well as having different sizes of needles can also have different sizes of cannula hub. The inclusion of a tapered/angled notch in a compressible material, and a securement strip allows for more universal use with different sizes and shapes of cannula. In particular, it allows different sizes and shapes of cannula/cannula hub to nestle into the space between the arms of the notch, with the needle (to be inserted into the skin) projecting away from the device from the wide end of the notch. In the placing of the cannula into the tapered notch of the cannula stabilization device, the taper allows the notch to be positioned and supported, without putting undue pressure on the application area of the patient. In addition, the tapered shape allows cannulas to be inserted at various different angles-the preferred angle of insertion may be a result of the location on the body (e.g., vein depths differ on arms, legs and the chest), the size of the patient (e.g., children or adults) and the skill of the practitioner (which can also affect the angle of insertion of a cannula).

The foam provides the device with flexibility, allowing it to conform to the shape of the body and the cannula/catheter hub, and therefore can be used on many different parts of the body.

The securement strip that is used to hold the cannula/catheter hub in position may include a repositionable adhesive to allow repositioning of the cannula/without requiring the device itself to be removed.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 shows a schematic view of an exemplary embodiment of a cannula stabilization device as described herein, with a cannula shown in place in the notch of the compressible pad.

FIG. 2 shows a top-down view from above the compressible pad of FIG. 1.

FIG. 3 shows a perspective view of another exemplary embodiment of a compressible pad that is elongated and forms the first and second portions of the device, and wherein an adhesive is disposed on the second (under) side of the compressible pad.

FIG. 4 shows a perspective view of a further exemplary embodiment of a compressible pad that is elongated and forms the first and second portions of the device, with a backing strip disposed on the second side of the compressible pad, and wherein an adhesive is disposed on the second (under) side of the backing strip.

FIGS. 5A to 5C show top-down views from above of exemplary embodiments of compressible pads with alternative notch configurations, in which:

FIG. 5A shows a multi-angle notch where the first angle made between the walls of the compressible pad that define the taper at the narrow end of the notch is smaller than the second angle made between the walls of the compressible pad that define the taper at the wide end of the notch;

FIG. 5B shows a multi-angle notch where the first angle made between the walls of the compressible pad that define the taper at the narrow end of the notch is larger than the second angle made between the walls of the compressible pad that define the taper at the wide end of the notch; and

FIG. 5C shows a compressible pad with a parabolic notch.

FIG. 6 shows a perspective view of still another exemplary embodiment of the cannula stabilization device as described herein, in which the compressible pad comprises two layers, with a stepped notch, where the lower layer (which, in use, would be closest to the skin of a patient) has a larger notch (longer length and width) than the upper layer.

### DETAILED DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

In general, a securement or stabilization device for holding an intravenous device such as a cannula or catheter to a patient is described herein. The stabilization device may be configured as a universal holding device, and be able to accommodate tubes of varying sizes and shapes, to eliminate the need for one-to-one pairing of the cannula or catheter to its corresponding shaped and sized holder, which is time consuming and poses the above-mentioned risks if an improper pairing is made.

Described hereinbelow are detailed features as well as optional features of the stabilization device. It is understood that any optional feature may be combined with any other optional feature.

In general, the stabilization device has: first and second portions, a first side and an opposing second side, and opposing first and second edges (i.e. a first edge and an opposing second edge). The compressible pad has a tapered notch, such that the notch has a wide end and a narrow end, the wide end being disposed at an edge of the compressible pad and at the first edge of the device, and between the first and second portions. In other words, the first and second portions of the device are located on either side of the notch of the compressible pad so as to form lateral arms. The second side of the device is typically the side of the device that, in use, would be closer to the skin of a patient than the first side of the device. The first and second edges of the device are faces of the device linking the first and second sides of the device. The device, when viewed from above the first side, may be of any suitable shape-for example elongated, rounded (e.g. circular or oval), or a regular shape, such as a triangle, rectangle or a square. In an embodiment, the device, when viewed from above the first side, is elongated, e.g. in the shape of a rectangle, such that the first and second portions extend away from the notch. In an embodiment, the device is elongated and in the form of a rectangle having a long edge and a short edge, with the wide end of the notch of the compressible pad being located on the long edge, preferably at or near the center of the long edge, with the first and second portions being located at either side of the notch.

### Compressible Pad

The stabilization device includes a compressible pad. The compressible pad has a tapered notch, such that the notch has a wide end and a narrow end, with the wide end being disposed at an edge of the compressible pad and at the first edge of the device. The tapered notch may have an angle of from 5 to 140 degrees, optionally 10 to 110 degrees, optionally 15 to 90 degrees, optionally 20 to 60 degrees, optionally 30 to 50 degrees, optionally 35 to 45 degrees, optionally about 40 degrees. In other words, the walls of the compressible pad that define the taper have the aforementioned angle. Alternatively, the tapered notch may be curved or parabolic. Alternatively, the tapered notch may comprise two or more angles, such that the angle between the walls of the compressible pad that define the taper changes from the narrow end to the wide end of the notch.

In an embodiment, the angle between the walls of the compressible pad (aside from any curvature at the narrow end) that define the taper remains constant along the length of the notch (from the narrow end to the wide end). In an embodiment, the angle between the walls of the compressible pad (aside from any curvature at the narrow end) that define the taper increases or decreases along the length of the notch (from the narrow end to the wide end). In an embodiment, the angle between the walls of the compressible pad (aside from any curvature at the narrow end) that define the taper increases or decreases along the length of the notch (from the narrow end to the wide end), and there may be at least two angles defined by straight portions of the walls that define the taper (i.e. the notch contain at least two angles). Where the notch contains two angles, a first angle made between the walls of the compressible pad that define the taper at the narrow end of the notch may be smaller than a second angle made between the walls of the compressible pad that define the taper at the wide end of the notch. Alternatively, where the notch contains two angles, a first angle made between the walls of the compressible pad that define the taper at the narrow end of the notch may be larger than a second angle made between the walls of the compressible pad that define the taper at the wide end of the notch. The tapered notch may have a first angle of from 5 to 140 degrees, optionally 10 to 110 degrees, optionally 15 to 90 degrees, optionally 20 to 60 degrees, optionally 30 to 50 degrees, optionally 35 to 45 degrees, optionally about 40 degrees, and a second angle of from 5 to 140 degrees, optionally 10 to 110 degrees, optionally 15 to 90 degrees, optionally 20 to 60 degrees, optionally 30 to 50 degrees, optionally 35 to 45 degrees, optionally about 40 degrees. The point at which the walls of the compressible pad that define the taper change angle (the vertex) may have a distance (measured in a direction perpendicular from the first edge of the compressible pad) from the narrow end of the notch from about 1 mm to about 50 mm, optionally from about 2 mm to about 40 mm, optionally from about 3 mm to about 30 mm, optionally from about 4 mm to about 20 mm, optionally from about 5 mm to about 15 mm, optionally from about 6 mm to about 10 mm, optionally about 8 mm.

These angles, together with the compressibility of the pad and the securement strip, allow most sizes and shapes of commercially available intravenous devices like cannulas or catheters to be accommodated in the device, and, moreover, held at different angles, to suit the particular needs/preferences of a patient. Some angles may provide more comfort to the patient and/or be more effective at administering/withdrawing fluids to/from a patient. Some angles of insertion may be a result of the location on the body (e.g., vein depths differ on arms, legs and the chest), the size of the patient (e.g., children or adults) and the skill of the practitioner (which can also affect the angle of insertion of a cannula). Accordingly, a multi-angle notch may be of benefit for certain installation sites and/or certain types of cannula hubs.

The length of the notch, as measured perpendicular from the wide end of notch to the narrow end of the notch, may be from about 5 mm to about 60 mm, optionally from about 6 to about 50 mm, optionally from about 7 to about 40 mm, optionally from about 8 to about 30 mm, optionally from about 10 mm to about 20 mm, optionally from about 12 mm to about 20 mm, optionally from about 13 mm to about 19 mm, optionally from about 14 mm to 18 mm, optionally from about 15 mm to 17 mm, optionally about 16 mm.

The depth of the compressible pad, i.e. the shortest distance from its first side to its second side, may be about 2 mm to 20 mm, optionally about 2 mm to 10 mm, optionally about 2 to about 9 mm, optionally about 3 mm to about 8 mm, optionally about 4 mm to about 7 mm, optionally about 6 mm.

The ratio of the depth of the compressible pad to the width of the wide end of the notch is 1:1 to 1:5, optionally 1:2 to 1:4, optionally about 1:2 to 1:3, optionally about 1:2 to 2:5.

The ratio of the depth of the compressible pad (in mm) to the angle of the notch (in degrees) is 1:1 to 1:20, optionally 1:1 to 1:10, optionally 1:2 to 1:10, optionally 1:3 to 1:9, optionally 1:4 to 1:8, optionally 1:5 to 1:7, optionally about 1:6.

The compressible pad may comprise multiple layers. The compressible pad may comprise multiple layers and each layer comprises a different notch geometry, e.g. notches having one or more of different lengths, different widths, a different angle between the narrow end and widest end, and notches with different numbers of angles. The multiple layers may create a “stair step” notch, e.g. a notch that is stepped in the direction from the first side of the compressible pad to the second side of the compressible pad. The compressible pad may comprise two layers, wherein a first compressible pad layer is disposed on the first side of the second compressible pad layer, and the second compressible pad layer comprises an indentation defining a notch, and the first compressible pad layer is continuous, but has a depression defining a notch in the area that the first layer overlies and is depressed into the indentation in the second compressible pad layer.

The notch may have a rounded narrow end (i.e. when viewed from above the first side). The rounded narrow end may have a radius of curvature of from about 0.5 mm to about 5 mm, optionally about 1 mm to about 4 mm, optionally about 1.5 mm to 3.5 mm, optionally about 2 mm to 3 mm.

The compressible pad is preferably formed from a resilient material, i.e. one that can be elastically deformed. Optionally, the compressible pad comprises a material selected from a foam, fabric, which may be a woven or a non-woven fabric, and a compressible non-porous material (e.g. a block of synthetic or natural rubber material). The compressible pad preferably comprises a closed cell foam. The compressible pad may comprise two or more layers of the same material, selected from a foam, fabric, which may be a woven or a non-woven fabric, and a compressible non-porous material (e.g. a block of synthetic or natural rubber material). The compressible pad may comprise two or more layers of different material selected from a foam, fabric, which may be a woven or a non-woven fabric, and a compressible non-porous material (e.g. a block of synthetic or natural rubber material). Optionally, the compressible pad may comprise a soft top layer and a more rigid bottom layer (“top” layer being the layer disposed further away, in use, from the skin of a patient than the “bottom” layer; and “soft” indicating a more compressible layer than a “hard” layer, i.e. a soft layer requiring less force to compress the same size foam a certain distance). The compressible pad may comprise two or more layers, each of which has a different compressibility or elasticity. Optionally, the compressible pad may comprise a top layer with lower compressibility or elasticity than the bottom layer. “Compressibility” and “elasticity” in the present context are synonymous, i.e. the compressibility and elasticity being measurable by the force required to compress the same size foam a certain distance, with a more compressible material requiring less force than a less compressible material. Compressibility may be tested using standard methods, including ASTM D3474C.

The compressible pad may comprise a polymer, which may be foamed or in non-porous form, wherein the polymer is selected from polyalkylene (e.g. polyethylene, polypropylene), halogenated polyalkylene (e.g. chlorosulphonated polyethylene), silicone, ethylene vinyl alcohol polymer, polychloroprene (neoprene), styrene butadiene, polystyrene, natural rubber, butyl rubber, nitrile rubber, hydrogenated nitrile, ethylene propylene diene, fluoroelastomer rubber, polyurethane, polyurea, polyimide, polyvinylchloride, hydrocolloid, hydrogel. The compressible pad preferably comprises a material selected from a polyethylene foam, a polyvinylchloride foam and a polyurethane foam, which may be microcellular polyurethane foam. The polyethylene may be low density polyethylene (LDPE).

### Adhesive

An adhesive is disposed on the second side of the device, at least on the first and second portions of the stabilization device, such as at the end portions. The adhesive, in use, will serve the purpose of adhering the device to the skin of a patient. The adhesive may be any skin adhesive. The adhesive may be selected from a skin adhesive selected from a silicone, an acrylic, a hydrocolloid, a hydrogel, a rubber adhesive and a polyurethane.

The skin adhesive may be a pressure-sensitive adhesive. A pressure sensitive adhesive may be defined as an adhesive that forms a bond with a substrate (e.g. human skin) when pressure is applied to the adhesive. Typically, no water, heat or solvent is required to adhere the adhesive to the substrate. The degree of bonding of a pressure sensitive adhesive is typically affected by the pressure used to apply the adhesive to a substrate surface (e.g. human skin).

The silicone adhesive may be a polymer comprising —(S1R₂)— repeating units. Each R of the —(SiR₂)— may be selected from alkyl or aryl. In an embodiment, the alkyl may be a C₁ to C₅ alkyl, such as methyl, ethyl, propyl, butyl and pentyl. The alkyl may be straight chain or branched. In an embodiment, the silicone is a polydimethylsiloxane polymer. The silicone may be a cross-linked silicone. The silicone may be a co-polymer, and the co-polymer may comprise a plurality of different types of silicone repeating units, and, in an embodiment, at least some of which are —(SiR₂)— repeating units, e.g. wherein each R is methyl, and other silicone units are present that allow cross-linking of the silicone. The silicone may be termed a silicone rubber. The silicone may be formed by addition-curing or condensation curing. The silicone may be formed by addition curing a silicone hydride and a vinyl-functional silicone, e.g. by using a suitable catalyst, such as platinum, forming —(CH₂—CH₂)— linkages between silicon atoms (the —(CH₂—CH₂)— being formed from the vinyl group of the vinyl-functional silicone and the hydride (H atom) of the hydride-functional silicone).

The skin adhesive, which may be a pressure sensitive adhesive, may comprise a hydrocolloid. The hydrocolloid may comprise hydrophilic particles and, in some embodiments, a rubber material. The hydrocolloid, or the hydrophilic particles, may be selected from naturally derived substances (such as silica, collagen, pectin, gelatin, starches, guar gum, gum arabic, xanthan gum, locust bean gum, gum karaya, alginic acid and its sodium or calcium salts) and synthetic substances (such as such as sodium carboxymethylcellulose (CMC), crosslinked sodium carboxymethylcellulose, crystalline sodium carboxymethyl cellulose, polyvinyl alcohol, polyvinyl pyrollidone, polyethylene glycols and polypropylene glycols, cross-linked dextran and starch-acrylonitrile graft copolymer, starch sodium polyacrylate, gluten, polymer of methyl vinyl ether and maleic acid and derivatives; polyvinyl pyrrolidone, polyethylene glycols, polypropylene glycols, metal and/or ammonium salts of polyacrylic acid and/or its copolymers, and metal or ammonium salts of polystyrene sulfonic acid). If hydrophilic particles are present in the skin adhesive, they may constitute from about 5%-40%, optionally 20%-40% by weight of the skin adhesive. The rubber material, in which hydrophilic particles may be dispersed, may be selected from natural and synthetic rubbers. The rubber material may comprise a rubber comprising a block co-polymer, e.g. a block copolymer comprising polydiene, such as isoprene, and a further monomer, e.g. styrene. They may be triblock or di-block co-polymers. The rubber material may comprise a material selected from styrene-isoprene-styrene copolymers, styrene-ethylene/butylene-styrene copolymers, polyisobutylene, and ethylene propylene diene monomer polymer (EPDM polymer).

The pressure-sensitive adhesive may comprise an acrylic polymer. An acrylic polymer may be defined as a polymer that has been formed from the polymerization of acrylate monomers. The acrylate monomers may be selected from (meth)acrylic acid and esters of (meth)acrylic acid, “(meth)” in (meth)acrylic acid indicates that the monomer may be acrylic acid or methacrylic acid. The pressure-sensitive adhesive may be formed from at least two different acrylate monomers, and optionally the at least two different acrylate monomers may be selected from esters of (meth)acrylic acid and esters of (meth)acrylic acid, and the different acrylate monomers may have different Tg temperatures from one another. The acrylate monomers may be of the formula:

R₁—C(═CH₂)—C(═O)—R₂

wherein R₁ is selected from H and Me, and R₂ is selected from H an alkyl group, e.g. a C₁-C₁₀ alkyl group. The acrylate monomers may be selected from n-butyl acrylate, 2-ethylhexyl acrylate, acrylic acid, vinyl acetate, and n-butyl methacrylate.

The adhesive, which may be a pressure-sensitive adhesive, may comprise a polyurethane. A polyurethane may be defined as the reaction product of a polyisocyanate and a polyalcohol (polyol), the reaction product having urethane linking groups. A polyisocyanate may be defined as a molecule with two or more isocyanate groups. A polyalcohol may be defined as a molecule with two or more hydroxyl groups. The reaction product is a polymer containing urethane linkages.

The isocyanate may be an aromatic isocyanate or an aliphatic isocyanate. The aromatic isocyanate may be selected from diphenylmethane diisocyanate (MDI) and toluene diisocyanate (TDI). The aliphatic isocyanate may be selected from hexamethylene diisocyanate (HDI) and isophorone diisocyanate (IPDI).

The polyol may, for example, be a polyol formed from polymerizing an alkylene oxide, such as propylene oxide (PO) or ethylene oxide (EO), or by polyesterification of a di-acid, such as adipic acid, with glycols, such as ethylene glycol or dipropylene glycol (DPG).

The rubber adhesive may be a synthetic or natural rubber adhesive. The rubber adhesive may be a hot melt adhesive. The rubber adhesive may comprise a styrene block copolymer, e.g. a styrene block co-polymer selected from styrene-butadiene-styrene (SBS), styrene-isoprene-styrene (SIS), styrene-ethylene/butylene-styrene (SEBS), styrene-ethylene/propylene(SEP).

A release liner may be present on the second surface of the adhesive. The release liner will serve to protect the adhesive during storage and transport of the device, and can be removed before adhering the device to the skin of a patient. The release liner may be selected from a polyester film, a polyolefin film, siliconized paper or a plastic laminated paper.

### Securement Strip

The stabilization device includes a securement strip. The securement strip is attached to the first side of the first portion of the stabilization device, wherein the securement strip can extend over the notch in the compressible pad to the second portion, and can be attached to the first side of the second portion of the stabilization device. The securement strip, in use, acts to secure the cannula (or catheter) in place on the device. The securement strip may comprise any suitable material. The securement strip may be permanently attached to the first side of the first portion of the device, and releasably affixable to the first side of the second portion of the device. The adhesives on the second side of the securement strip may be suitably chosen to allow for a desired level of adhesion. The adhesive may be selected from a skin adhesive selected from a silicone, an acrylic, a hydrocolloid, a rubber adhesive a hydrogel and a polyurethane, e.g. as described in the paragraphs above. Where the securement strip is to be releasably adhered to the first side of the second portion of the device, the adhesive on the second side of the securement strip that adheres to the first side of the second portion of the device may be a pressure sensitive adhesive, for example selected from a silicone, an acrylic, a hydrocolloid, a hydrogel, a rubber adhesive and a polyurethane, e.g. as described in the paragraphs above.

The securement strip may be any flexible material, which may or may not be elasticated. The securement strip may comprise a continuous web of material, e.g. a sheet of material, which may be a polymeric material. Alternatively, or in addition, the securement strip may comprise a fabric, which may be selected from a woven fabric, a non-woven fabric and a netting, a foam, which may be an open-or closed-cell foam, and a paper.

The securement strip may comprise a material selected from a natural or synthetic polymer. Natural polymers may be selected from cellulosic polymers, e.g. cotton and wool (e.g. if the securement strip is in the form of a fabric), and wood fibres (e.g. if the securement strip is in the form of a paper). The synthetic polymers may be selected from polyalkylenes (such as polyethylene and polypropylene), polyvinylchloride, polyesters (such as polyethylene terepthalate (PET) and polybutylene terepthalate (PBT)), polyamides, polystyrene, acrylonitrile, polylactic acid, polyamides, polycarbonates, and silicones.

If the securement strip is in the form of or comprises a foam, the foam material may be selected from a polymer selected from polyalkylene (e.g. polyethylene, polypropylene), halogenated polyalkylene (e.g. chlorosulphonated polyethylene), silicone, ethylene vinyl alcohol polymer, polychloroprene (neoprene), styrene butadiene, polystyrene, natural rubber, butyl rubber, nitrile rubber, hydrogenated nitrile, ethylene propylene diene, fluoroelastomer rubber, polyurethane, polyurea, polyimide, polyvinylchloride foam. Both the compressible pad and the securement strip may comprise a foam and the foam of the compressible pad may be same as or different from that of the securement strip.

The securement strip may be transparent, translucent or opaque.

The securement strip may have a thickness, as measured from the first side to the second side, of from about 0.005 mm to about 10 mm, optionally from about 0.005 mm to about 8 mm, optionally from about 0.005 mm to about 5 mm, optionally from about 0.005 mm to about 3 mm, optionally from about 0.005 mm to about 1.0 mm, optionally from about 0.005 mm to about 0.3 mm.

### Backing Strip

In an embodiment, the stabilization device has a backing strip. The backing strip may be located on a second side of the compressible pad. The backing strip may form the first and second portions, first side and opposing second side, and first and second edges linking first and second sides of the stabilization device, and wherein the compressible pad is disposed on the backing strip. The backing strip may extend beyond the compressible pad, such that the first and second portions of the device are formed primarily by the backing strip. In an alternative embodiment, the compressible pad may cover the backing strip entirely, so the compressible pad on either side of the notch may itself have the first and second portions.

The backing strip may be a single layer or a plurality of layers. If a plurality of layers are present, they may be of the same or different materials as one another.

The backing strip may be any flexible material, which may or may not be elasticated. The backing strip is preferably conformable to the contours of a human or animal body. The backing strip may comprise a continuous web of material, e.g. a sheet of material, which may be a polymeric material. Alternatively, or in addition, the backing strip may comprise a material selected from a woven fabric, a non-woven fabric and a netting, a foam, which may be an open-or closed-cell foam, and a paper.

The backing strip may comprise a material selected from a natural or synthetic polymer. Natural polymers may be selected from cellulosic polymers, e.g. cotton and wool (e.g. if the backing strip is in the form of a fabric), and wood fibres (e.g. if the backing strip is in the form of a paper). The synthetic polymers may be selected from polyalkylenes (such as polyethylene and polypropylene), polyvinylchloride, polyesters (such as polyethylene terepthalate (PET) and polybutylene terepthalate (PBT)), polyamides, acrylonitrile, polylactic acid, polyamides, polycarbonates and silicones. The backing strip may comprise a hydrocolloid or a hydrogel.

The backing strip may comprise one or more layers comprising a polymeric material, e.g. one or more layers of a polymeric film. The backing strip may be selected from a polycellulose film, a polyurethane film, a polyester film, a polyethylene film, a polyolefin film or any mixture thereof.

If the backing strip is in the form of or comprises a foam, the foam material may be selected from a polymer selected from polyalkylene (e.g. polyethylene, polypropylene), halogenated polyalkylene (e.g. chlorosulphonated polyethylene), silicone, ethylene vinyl alcohol polymer, polychloroprene (neoprene), styrene butadiene, polystyrene, natural rubber, butyl rubber, nitrile rubber, hydrogenated nitrile, ethylene propylene diene, fluoroelastomer rubber, polyurethane, microcellular urethane, hydrocolloid, hydrogel, polyurea, polyimide, polyvinylchloride foam. Both the compressible pad and the backing strip may comprise a foam and the foam of the compressible pad may be same as or different from that of the backing strip.

The backing strip may be transparent, translucent or opaque.

The backing strip may have a thickness, as measured from the first side to the second side, of from about 0.005 mm to about 3 mm, optionally from about 0.005 mm to about 2 mm, optionally from about 0.005 mm to about 1.0 mm, optionally about 0.005 mm to about 0.3 mm.

In an exemplary method of using the stabilization device to secure a cannula to a patient, the following steps may be involved:
a. inserting a needle of a cannula into a blood vessel of a patient,
b. adhering a stabilization device as described herein on the skin of a patient, with the adhesive on the second side of the device contacting the skin of the patient,
c. placing the cannula into the tapered notch of the stabilization device, such that the needle extends from away from the wide end of the notch, and
d. extending the securement strip over the cannula and the notch, such that it is adhered to the first side of both the first and second portions of the stabilization device.
   In the method, steps a, b and c may be carried out in any order, e.g. first a, then b, then c, or first b, then a, then c, or first c, then a, then b. Step d is preferably carried out once the cannula is in the desired position on the device and the needle of the device inserted into a blood vessel and the device is adhered to the skin of a patient.

The patient may be a human or animal. In the step of placing of the cannula into the tapered notch of the cannula stabilization device, the taper allows the notch to be positioned and supported, without putting undue pressure on the application area of the patient.

The cannula can comprise a needle and a cannula hub, from which the needle extends. A tubing may extend from the hub. The tubing may form part of the cannula during insertion or may be affixed to the cannula after insertion (e.g. after steps a to d of the method are complete). The size of the needle and/or tubing of the cannula may be from 14 to 26 gauge (2 mm to 0.46 mm). The cannula may be a peripheral venous catheter, e.g. having a valve for insertion of drugs with a syringe and a portion that allows connection to an intravenous infusion line or tubing. The cannula may be inserted into a blood vessel selected from a vein and an artery. Once in place on the device and inserted into the blood vessel of a patient, the cannula may be used to administer or withdraw liquids through blood vessels in a human or animal body.

Examples of the stabilization device will now be further described with reference to the accompanying non-limiting Figures, in which:

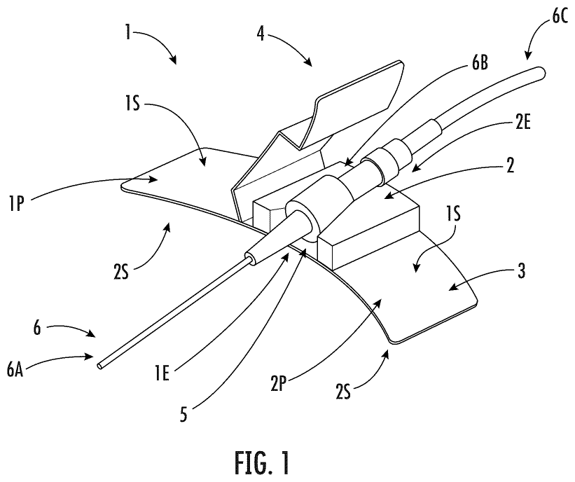

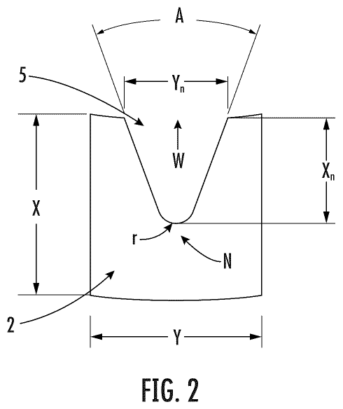

FIG. 1 shows an exemplary embodiment of a cannula stabilization device (1). The device has a compressible pad (2), a backing strip (3), and a securement strip (4). In this embodiment, the backing strip (3), and hence the device, has first and second portions (1P, 2P), a first side (1S) and an opposing second side (2S), and a first edge and opposing second edge (1E, 2E) linking first and second sides (1S, 2S). The compressible pad (shown from above in FIG. 2) has a tapered notch (5), such that the notch has a wide end (W) and a narrow end (N), the wide end (W) being disposed at an edge of compressible pad and at the first edge (1E) of the device. The compressible pad is made of foamed LDPE, and is adhered to the backing strip by an adhesive (not shown), which may be an acrylic adhesive. The securement strip (4) is attached to the first side (1S) of the first portion (1P) of the backing strip (3). The securement strip (4) can extend over the notch (5) in the compressible pad to the second portion (2P) of the backing strip, and can be attached to the first side (1S) of the second portion (2P) of the backing strip. An adhesive (not shown) is disposed on the second side (2S) of both the first and second portions (1P, 2P) of the backing strip, and may be disposed under the compressible pad, i.e. between first and second portions. The adhesive may be any skin adhesive, preferably a pressure sensitive skin adhesive. The adhesive may be an acrylic adhesive.

A cannula (6) is seen in place on the device (1). The cannula has a needle (6A), a cannula hub (6B) and cannula tubing (6C). The needle is for insertion into a blood vessel of a patient. The cannula hub sits in the notch (5) of the compressible pad (2), with the needle of the cannula extending away from the wide end (W) of the notch. In use, once the needle of the cannula has been inserted into a blood vessel—either a vein or an artery—and in a desired position in the notch of the device, the securement strip would extend over the cannula from the first side of the first portion (1S) to the first side of the second portion, to which it is releasable affixed. This keeps the cannula in the desired position, and minimizes the needle pistoning in and out of the skin, any movement of the needle and the cannula causing bruising, or dislodgement of the cannula.

FIG. 2 shows, from above the first side of the device, the compressible pad (2) of FIG. 1. The pad (2) has the notch (5) having the wide end (W) and narrow end (N). The angle (A) of the taper formed by the sides of the notch running from the narrow end (N) to the wide end (W). In the Figure, angle (A) is 40°, which has been found to be a suitable angle to accommodate most, if not all, cannula hubs and allow them to be positioned at different angles, but nevertheless remain secure on the device. The angle A can be varied, as described elsewhere herein. Also shown in FIG. 2 are lateral dimensions of the compressible pad and the notch, the lateral dimensions being defined as the length (X) of the compressible pad and the length (Xₙ) of the notch and the width (Y) of the compressible pad and the width (Yₙ) of the notch. The notch has a rounded narrow end (N), with the radius of curvature of the rounded narrow end being denoted by r in the Figure. In an embodiment, X is 26.9 mm, Y is 25.4 mm, Xₙ is 15.7 mm, Yₙ is 15.0 mm, and r is 2.54 mm. The compressible pad also has a depth (z), not shown in FIG. 2, of about 6.35 mm.

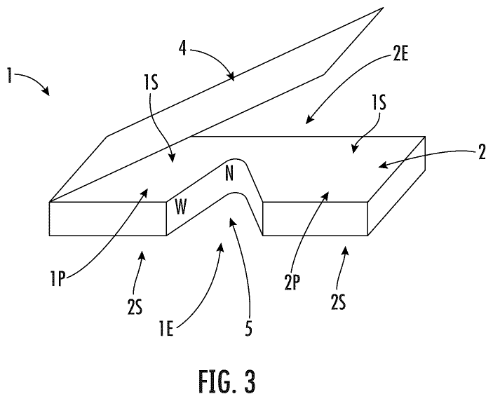

FIG. 3 shows another example of a cannula stabilization device (1), wherein the compressible pad is elongated and forms the first and second portions of the device, and wherein an adhesive is disposed on the second (under) side of the compressible pad. The device has a compressible pad (2) and a securement strip (4). In this embodiment, the compressible pad (2) is elongated and has, and hence the device has, first and second portions (1P, 2P), a first side (1S) and an opposing second side (2S), and first and second edges (1E, 2E) linking first and second sides (1S, 2S). The compressible pad (2) has a tapered notch (5), such that the notch has a wide end (W) and a narrow end (N), the wide end (W) being disposed at the first edge (1E) of the device. The securement strip (4) is attached to the first side (1S) of the first portion (1P) of the compressible pad (2). The securement strip (4) can extend over the notch (5) in the compressible pad (2) to the second portion (2P) of the compressible pad (2), and can be attached to the first side (1S) of the second portion (2P) of the compressible pad (2). An adhesive (not shown) is disposed on the second side (2S) of both the first and second portions (1P, 2P) of the compressible pad (2).

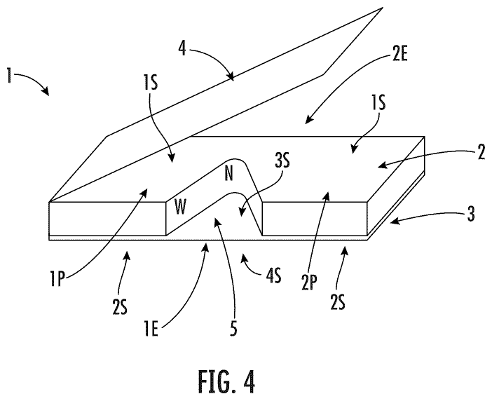

FIG. 4 shows a further example of a cannula stabilization device (1), wherein the compressible pad is elongated and forms the first and second portions of the device, with a backing strip disposed on the second side of the compressible pad, and wherein an adhesive is disposed on the second (under) side of the backing strip. The device has a compressible pad (2), a backing strip (3), and a securement strip (4). In this embodiment, the compressible pad (2) is elongated and has, and hence the device has, first and second portions (1P, 2P), a first side (1S) and an opposing second side (2S), and first and second edges (1E, 2E) linking first and second sides (1S, 2S). The compressible pad (2) has a tapered notch (5), such that the notch has a wide end (W) and a narrow end (N), the wide end (W) being disposed at the first edge (1E) of the device. The securement strip (4) is attached to the first side (1S) of the first portion (1P) of the compressible pad (2). The securement strip (4) can extend over the notch (5) in the compressible pad (2) to the second portion (2P) of the compressible pad (2), and can be attached to the first side (1S) of the second portion (2P) of the compressible pad (2). In this embodiment, the backing strip (3) has a first side (3S) and opposing second side (4S). The first side (3S) of the backing strip (3) is disposed on the second side (2S) of the compressible pad (2). The compressible pad (2) completely covers the backing strip (3), aside from the notch of the compressible pad. An adhesive (not shown) is disposed on the second side (4S) of the backing strip (3).

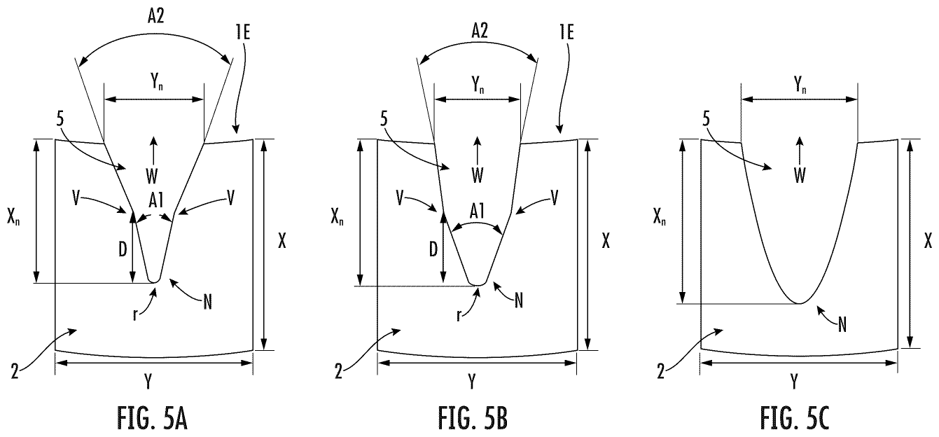

FIG. 5A shows, from above the first side of the device, another example of the compressible pad (2) of FIG. 1. The compressible pad (2) has the notch (5) having the wide end (W) and narrow end (N). The notch has a first angle (A1) and a second angle (A2), where the first angle (A1) is smaller than the second angle (A2). The angles can be varied as described herein. Also shown in FIG. 5A is the distance (D) (measured in a direction perpendicular from the first edge (1E) of the compressible pad) from the narrow end of the notch to the point where the notch angle changes (V). The distance (D) can be varied as described herein. Also shown in FIG. 5A are lateral dimensions of the compressible pad and the notch, the lateral dimensions being defined as the length (X) of the compressible pad and the length (Xₙ) of the notch and the width (Y) of the compressible pad and the width (Yₙ) of the notch. The notch has a rounded narrow end (N), with the radius of curvature of the rounded narrow end being denoted by r in the Figure.

FIG. 5B shows, from above the first side of the device, another example of the compressible pad (2) of FIG. 1. The compressible pad (2) has the notch (5) having the wide end (W) and narrow end (N). The notch has a first angle (A1) and a second angle (A2), where the first angle (A1) is larger than the second angle (A2). The angles can be varied as described herein. Also shown in FIG. 5B is the distance (D) (measured in a direction perpendicular from the first edge (1E) of the compressible pad) from the narrow end of the notch to the point where the notch angle changes (V). The distance (D) can be varied as described herein. Also shown in FIG. 5B are lateral dimensions of the compressible pad and the notch, the lateral dimensions being defined as the length (X) of the compressible pad and the length (Xₙ) of the notch and the width (Y) of the compressible pad and the width (Yₙ) of the notch. The notch has a rounded narrow end (N), with the radius of curvature of the rounded narrow end being denoted by r in the Figure.

FIG. 5C shows, from above the first side of the device, another example of the compressible pad (2) of FIG. 1. The compressible pad (2) has the notch (5) having the wide end (W) and narrow end (N). The notch is parabolic in shape. Also shown in FIG. 5B are lateral dimensions of the compressible pad and the notch, the lateral dimensions being defined as the length (X) of the compressible pad and the length (Xₙ) of the notch and the width (Y) of the compressible pad and the width (Yₙ) of the notch.

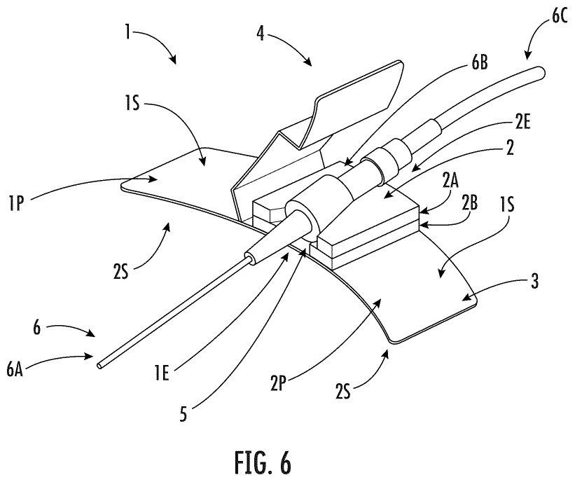

FIG. 6 shows a further example of a cannula stabilization device (1). This cannula stabilization device (1) shows a “stair step” notch, e.g. a notch that is stepped in the direction from the first side of the compressible pad to the second side of the compressible pad. In this example, the compressible pad (2) comprises two layers (2A, 2B), with a stepped notch (5), formed by having a lower layer (2A, which, in use, would be closest to the skin of a patient) has a larger notch (longer length (Xₙ) and width (Yₙ)) than the upper layer (2B). All other components are the same as in FIG. 1 (and given the same labels in the Figure). This cannula stabilization device (1) may be even more versatile than others in accommodating different sizes of a cannula, while, in use, holding the cannula securely in place on a patient's skin.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, according to one aspect, in a 1^{st} embodiment, a stabilization device for a cannula is provided. The stabilization device can comprise first and second portions, a first side and an opposing second side, and a first edge and opposing second edge. The stabilization device can also comprise a compressible pad having a tapered notch, the notch having a wide end and a narrow end, wherein the wide end is disposed at an edge of the compressible pad and at the first edge of the device between the first and second portions. An adhesive may be disposed on the second side at least on the first and second portions of the stabilization device. A securement strip may also be attached to the first side of the first portion of the stabilization device, wherein the securement strip is configured to extend over the notch in the compressible pad to the second portion, and is attachable to the first side of the second portion of the stabilization device.

A 2^{nd} embodiment is the 1^{st} embodiment of the stabilization device, wherein the stabilization device may comprise a backing strip, and the first and second portions, first side and opposing second side, and first and opposing second edges of the stabilization device are part of the backing strip.

A 3^{rd} embodiment is any combination of the first 2 embodiments, wherein the compressible pad may be disposed on the first side of the backing strip, and the adhesive may be disposed on the second side of the backing strip for adhesion to a skin of a patient.

A 4^{th} embodiment is any combination of the first 3 embodiments, wherein the first and second portions, first side and the opposing second side, and first and second edges of the stabilization device are part of the compressible pad, and the adhesive is disposed on the second side of the first and second portions of the compressible pad for adhesion to skin of a patient.

A 5^{th} embodiment is any combination of the first 4 embodiments, wherein the notch may have an angle of between 5 to 140 degrees, or between 30 to 60 degrees, or between 30 to 50 degrees.

A 6^{th} embodiment is any combination of the first 5 embodiments, wherein the notch may have a rounded narrow end.

A 7^{th} embodiment is any combination of the first 6 embodiments, wherein the compressible pad may have a depth and a width, and the ratio of the depth of the compressible pad to the width of the wide end of the notch can be 1:1 to 1:5, or 1:2 to 1:4, or 1:3.

An 8^{th} embodiment is any combination of the first 7 embodiments, wherein the compressible pad can comprise a material selected from foam, fabric and a compressible non-porous material.

A 9^{th} embodiment is any combination of the first 8 embodiments, wherein the materials of the compressible pad can comprise a polymer selected from polyalkylene (e.g. polyethylene, polypropylene), halogenated polyalkylene (e.g. chlorosulphonated polyethylene), silicone, ethylene vinyl alcohol polymer, polychloroprene (neoprene), styrene butadiene, polystyrene, natural rubber, butyl rubber, nitrile rubber, hydrogenated nitrile, ethylene propylene diene, fluoroelastomer rubber, polyurethane (e.g. a microcellular polyurethane), polyurea, polyimide, polyvinylchloride, hydrocolloid and hydrogel.

A 10^{th} embodiment is any combination of the first 9 embodiments, wherein the compressible pad can comprise a closed cell foam.

An 11^{th} embodiment is any combination of the first 10 embodiments, wherein the backing strip can comprise a continuous web of material, a fabric, a foam, and a paper.

A 12^{th} embodiment is any combination of the first 11 embodiments, wherein the backing strip can comprise a polymeric material.

A 13^{th} embodiment is any combination of the first 12 embodiments, wherein the adhesive can be selected from a silicone, an acrylic, a hydrocolloid, a hydrogel, a rubber adhesive and a polyurethane.

A 14^{th} embodiment is any combination of the first 13 embodiments, wherein the stabilization device can also include a release liner on the adhesive.

A 15^{th} embodiment is any combination of the first 14 embodiments, wherein the compressible pad can comprise two layers.

A 16^{th} embodiment is any combination of the first 15 embodiments, wherein the tapered notch can be a stepped notch.

According to a second aspect, in a 1^{st} embodiment, a method of securing a cannula to a patient is provided. The method may include the steps of inserting a needle of a cannula into a blood vessel of a patient, adhering the stabilization device on a skin of the patient, placing the cannula into the tapered notch of the stabilization device such that the needle extends from away from the wide end of the notch, and extending the securement strip over the cannula and the notch, such that it is adhered to the first side of both the first and second portions of the stabilization device.

A 2^{nd} embodiment is the 1^{st} embodiment of the method, wherein the cannula can comprise a cannula hub from which the needle of the cannula extends, and the cannula hub can be placed in the tapered notch.

A 3^{rd} embodiment is any combination of the first 2 embodiments, wherein the cannula can comprise tubing, and the tubing can be placed in the tapered notch.

What is claimed is:

1. A stabilization device for a cannula, comprising:
first and second portions, a first side and an opposing second side, and a first edge and opposing second edge;
a compressible pad having a tapered notch, the notch having a wide end and a narrow end, wherein the wide end is disposed at an edge of the compressible pad and at the first edge of the device between the first and second portions;
an adhesive disposed on the second side at least on the first and second portions of the stabilization device; and
a securement strip attached to the first side of the first portion of the stabilization device, wherein the securement strip is configured to extend over the notch in the compressible pad to the second portion, and is attachable to the first side of the second portion of the stabilization device.

2. The stabilization device according to claim 1, wherein the device comprises a backing strip, and the first and second portions, first side and opposing second side, and first and opposing second edges of the stabilization device are part of the backing strip.

3. The stabilization device according to claim 2, wherein the compressible pad is disposed on the first side of the backing strip, and the adhesive is disposed on the second side of the backing strip for adhesion to a skin of a patient.

4. The stabilization device according to claim 1, wherein the first and second portions, first side and the opposing second side, and first and second edges of the stabilization device are part of the compressible pad, and the adhesive is disposed on the second side of the first and second portions of the compressible pad for adhesion to skin of a patient.

5. The stabilization device according to any claim 1, wherein the notch has an angle of between 5 to 140 degrees.

6. The stabilization device according to claim 1, wherein the notch has a rounded narrow end.

7. The stabilization device according to claim 1, wherein the compressible pad has a depth and a width, and the ratio of the depth of the compressible pad to the width of the wide end of the notch is 1:1 to 1:5.

8. The stabilization device according to claim 1, wherein the compressible pad comprises a material selected from foam, fabric and a compressible non-porous material.

9. The stabilization device according to claim 8, wherein the materials of the compressible pad comprises a polymer selected from polyalkylene (e.g. polyethylene, polypropylene), halogenated polyalkylene (e.g. chlorosulphonated polyethylene), silicone, ethylene vinyl alcohol polymer, polychloroprene (neoprene), styrene butadiene, polystyrene, natural rubber, butyl rubber, nitrile rubber, hydrogenated nitrile, ethylene propylene diene, fluoroelastomer rubber, polyurethane (e.g. a microcellular polyurethane), polyurea, polyimide, polyvinylchloride, hydrocolloid and hydrogel.

10. The stabilization device according to claim 8, wherein the compressible pad comprises a closed cell foam.

11. The stabilization device according to claim 2, wherein the backing strip comprises a continuous web of material, a fabric, a foam, and a paper.

12. The stabilization device according to claim 2, wherein the backing strip comprises a polymeric material.

13. The stabilization device according to claim 1, wherein the adhesive is selected from a silicone, an acrylic, a hydrocolloid, a hydrogel, a rubber adhesive and a polyurethane.

14. The stabilization device according to claim 1, wherein the compressible pad comprises two layers.

15. The stabilization device according to claim 14, wherein the tapered notch is a stepped notch.

16. The stabilization device according to claim 1, further including a release liner on the adhesive.

17. A method of securing a cannula to a patient, comprising:
inserting a needle of a cannula into a blood vessel of a patient,
adhering the stabilization device according to claim 1 on a skin of the patient,
placing the cannula into the tapered notch of the stabilization device such that the needle extends from away from the wide end of the notch, and
extending the securement strip over the cannula and the notch, such that it is adhered to the first side of both the first and second portions of the stabilization device.

18. The method according to claim 15, wherein the cannula comprises a cannula hub from which the needle of the cannula extends, and the cannula hub is placed in the tapered notch.

19. The method according to claim 1, wherein the cannula comprises tubing, and the tubing is placed in the tapered notch.
